(19) United States
(12) Patent Application Publication
(10) Pub. No.: US 2005/0186611 A1
(43) Pub. Date: Aug. 25, 2005

(54) Method and apparatus for manufacturing microarray

(76) Inventors: Jong-myeon Park, Seoul (KR); Yong-soo Oh, Seongnam-si (KR); Han-sung Ko, Seongnam-si (KR); Keon Kuk, Yongin-si (KR); Young-nam Kwon, Gunpo-si (KR)

Correspondence Address: CANTOR COLBURN, LLP, 55 GRIFFIN ROAD SOUTH, BLOOMFIELD, CT, 06002, US

(21) Appl. No.: 11/064,707

(22) Filed: Feb. 24, 2005

(30) Foreign Application Priority Data
Feb. 25, 2004 (KR) 10-2004-0012539

(51) Int. Cl. C12Q 1/68; B41J 2/01; B05D 3/00; C12M 1/34

(52) U.S. Cl. 435/6; 435/287.2; 427/2.11; 347/1

(57) ABSTRACT

A method of manufacturing a microarray by non-contact spotting a biomolecular solution onto a surface of a solid support using a thermally driven print head. The thermally driven print head used to manufacture the microarray includes a discharge chamber filled with the biomolecular solution to be spotted and being substantially semispherical.

### CROSS-REFERENCE TO RELATED PATENT APPLICATIONS

This application claims the benefit of Korean Patent Application No. 10-2004-0012539, filed on Feb. 25, 2004, in the Korean Intellectual Property Office, the disclosure of which is incorporated herein in its entirety by reference.

### BACKGROUND OF THE INVENTION

1. Field of the Invention

The present invention relates to a method and apparatus for manufacturing a microarray, and more particularly, to a method and apparatus for manufacturing a microarray using a thermally driven inkjet print head.

2. Description of the Related Art

Microarrays or biochips refer to microchips in which biomolecules such as a number of oligonucleotides or peptides are immobilized on a surface of a solid support in a predetermined pattern. Such microarrays occupy an important position in the bioengineering field such processes as disease diagnosis, drug development, nucleotide sequence identification, etc.

In a conventional method of manufacturing a microarray, a tip portion of a pin is dipped in a biomolecular solution, drawn therefrom, and brought in contact with a surface of a solid support. However, due to the physical contact between the pin and the solid support, both the tip portion of the pin and the surface of the solid support deform, thereby deteriorating the uniformity of microarrays on the support. In another conventional method of manufacturing a microarray, biomolecules are attached to a solid support by spotting a biomolecular solution onto a solid substrate in a non-contact manner using a piezoelectric inkjet print head. However, the method of manufacturing a microarray using the piezoelectric inkjet print head is complicated and has low reproducibility.

### SUMMARY OF THE INVENTION

The present invention provides a method and apparatus for manufacturing a microarray by non-contact spotting a biomolecular solution onto a surface of a solid substrate using a thermally driven inkjet print head.

According to an aspect of the present invention, there is provided a method of manufacturing a microarray by non-contact spotting a biomolecular solution onto a surface of a solid support using a thermally driven inkjet print head that comprises a discharge chamber filled with the biomolecular solution to be spotted and being substantially semispherical.

The thermally driven inkjet print head may comprise: a substrate in which the discharge chamber, a manifold storing the biomolecular solution to be supplied to the discharge chamber, and a channel connecting the discharge chamber and the manifold are formed; a nozzle plate disposed on the substrate and having nozzles through which the biomolecular solution is discharged from the discharge chamber;
a heater formed around each of the nozzles; and an electrode electrically connected to the heater to supply current to the heater.

The method of manufacturing a microarray may comprise: supplying current to the heater through the electrode; heating the biomolecular solution in the discharge chamber using heat generated by the heater to generate and expand bubbles; and discharging the biomolecular solution from the discharge chamber onto the surface of the solid support through the nozzles by the expansive force of the bubbles.

In the thermally driven inkjet print head, a plurality of discharge chambers, a plurality of manifolds, and a plurality of channels may be formed in the substrate. The substrate may be formed of silicon. The nozzle plate may be formed of a silicon oxide layer or a silicon nitride layer. The heater may have an annular shape and surround a corresponding nozzle in the nozzle plate. The heater may be formed of polycrystalline silicon or a tantalum-aluminum alloy. The electrode may be formed of aluminum or an aluminum alloy. A nozzle guide extending toward the discharging chamber may be additionally formed along a sidewall of each of the nozzles.

The biomolecular solution may contain an oligonucleotide, a protein, or cDNA as biomolecules. The solid support may be formed of glass or a silicon wafer. The surface of the solid support may be coated with at least one material selected from the group consisting of amine, aldehyde, and epoxy.

According to anther aspect of the present invention, there is provided a thermally driven inkjet print head for manufacturing a microarray by non-contact spotting a biomolecular solution onto a surface of a solid support, the thermally driven inkjet print head comprising: a substrate in which a plurality of discharge chambers filled with the biomolecular solution to be spotted, a plurality of manifolds storing the biomolecular solution to be supplied to the discharge chambers, and a plurality of channels respectively connecting the discharge chambers and the manifolds are formed; a nozzle plate disposed on the substrate and having a plurality of nozzles that respectively correspond to the discharge chambers; a plurality of heaters respectively formed around the nozzles; and a plurality of electrodes respectively electrically connected to the heaters to supply current to the heaters.

In the thermally driven inkjet print head, the discharge chambers may be substantially semispherical. The substrate may be formed of silicon. The nozzle plate may be formed of a silicon oxide layer or a silicon nitride layer. The heaters may have annular shapes and respectively surround the nozzles in the nozzle plate. The heaters may be formed of polycrystalline silicon or a tantalum-aluminum alloy. The electrodes may be formed of aluminum or an aluminum alloy. A nozzle guide extending toward a corresponding discharging chamber may be formed along a sidewall of each of the nozzles.

### BRIEF DESCRIPTION OF THE DRAWINGS

The above and other features and advantages of the present invention will become more apparent by describing in detail exemplary embodiments thereof with reference to the attached drawings in which:

FIG. 1 is a plane view illustrating the arrangement of nozzles in a thermally driven inkjet print head used to manufacture a microarray according to an embodiment of the present invention;

FIG. 2 illustrates a structure of a portion of the thermally driven inkjet print head in FIG. 1;

FIG. 3 is a sectional view illustrating a unit structure of the thermally driven inkjet print head in FIG. 1;

FIGS. 4A and 4B illustrate the results of gel electrophoresis on DNA before and after being discharged from the thermally driven inkjet print head used to manufacture a microarray according to the embodiment of the present invention; and

FIGS. 5A and 5B illustrate the results of analysing DNA using high performance liquid chromatography (HPLC) before and after being discharged from the thermally driven inkjet print head used to manufacture a microarray according to the present invention.

### DETAILED DESCRIPTION OF THE INVENTION

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. In the drawings, like reference numerals denote like elements, and the thicknesses of layers and regions are exaggerated for clarity. It will also be understood that when a layer is referred to as being “on” another layer or substrate, it can be directly on the other layer or substrate, or intervening layers may also be present.

In general, an inkjet print head is an apparatus for printing an image in a predetermined color by discharging microdroplets of ink for printing onto a desired position on printing paper. Such inkjet print heads can be classified into either a thermally driven inkjet print head, which generates bubbles of ink using a heat source and discharges droplets of ink by the expansive force of the bubbles, or a piezoelectric inkjet print head, which discharges droplets of ink due to the pressure generated as a result of deformation of a piezoelectric body.

A mechanism of discharging droplets of ink in the thermally driven inkjet print head will described in detail below. As current is supplied as pulses to a heater made of a heating resistor, the heater generates heat, and ink adjacent to the heater is spontaneously heated to about 300° C. As a result, the ink boils. The ink bubbles resulting from the boiling expand and increase the internal pressure of an ink chamber filled with the ink. The ink is discharged as droplets through nozzles outside the ink chamber.

Such thermally driven inkjet print heads are classified into a top-shooting type, a side-shooting type, or a back-shooting type according to the direction in which ink bubbles swell and the direction in which ink droplets are discharged. In a top-shooting type inkjet print head, ink bubbles swell and ink droplets are discharged in the same direction. In a side-shooting type inkjet print head, the direction in which ink bubbles swell and the direction in which ink droplets are discharged are perpendicular to each other. In a back-shooting type inkjet print head, the direction in which ink bubbles swell and the direction in which ink droplets are discharged are opposite to each other.

According to the present invention, a microarray is manufactured by non-contact spotting a biomolecular solution onto a surface of a solid support using a back-shooting type thermally driven inkjet print head among the above-described three types of inkjet print head. The biomolecular solution may contain biomolecules such as an oligonuelotide, a protein, cDNA, etc., according to the use of the microarray. The solid support may be made of glass, a silicon wafer, etc. The solid support is coated with chemical substance that can chemically bind with amine, aldehyde, epoxy, etc.

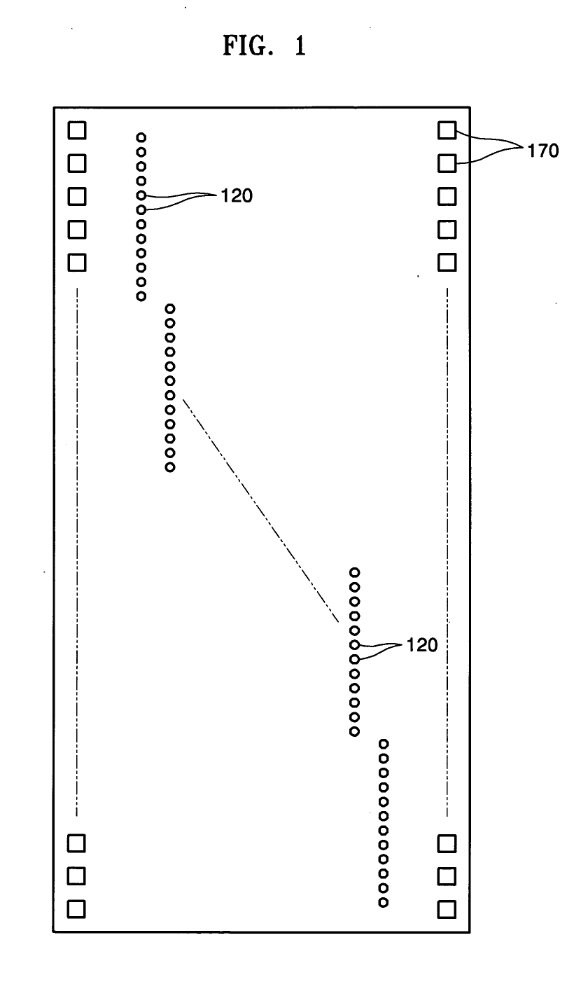

FIG. 1 is a plane view of a thermally driven inkjet print head used to manufacture a microarray according to an embodiment of the present invention.

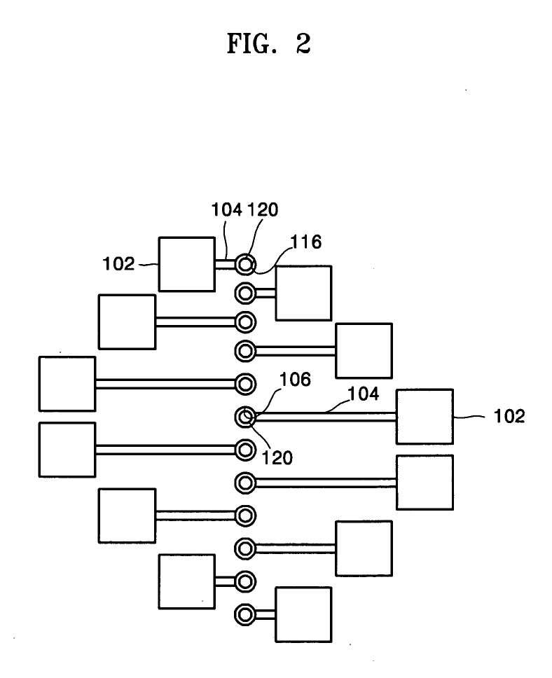

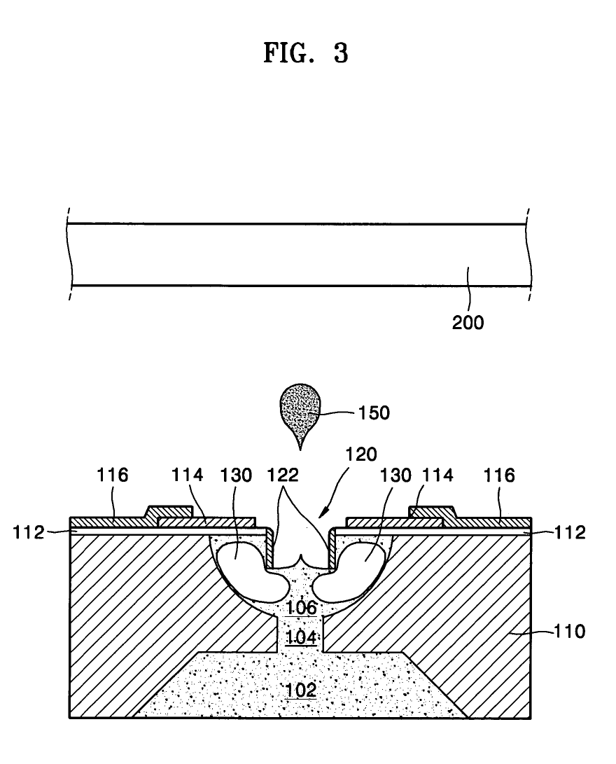

Referring to FIG. 1, a plurality of nozzles 120 through which a predetermined biomolecular solution is discharged are arranged on a surface of a thermally driven inkjet print head, and pads 170, which are electrically connected to electrodes 116 (see FIG. 2) for supplying current to a heater 114 (see FIG. 3), are disposed along two edges of the surface of the inkjet print head. In FIG. 1, 12 nozzles 120 are arranged in 11 rows at a constant interval. The nozzles 120 have an interval of about 80 μm therebetween. Using the inkjet print head having the nozzles 120 arranged as described above, a microarray on which biomolecules are integrated at 132 sites on a solid support can be manufactured. The arrangement of the nozzles 120 or the interval between the nozzles 120 can be varied according to the type of a microarray to be manufactured.

FIG. 2 illustrates a structure of a portion of the thermally driven inkjet print head in FIG. 1. FIG. 3 is a sectional view illustrating a unit structure of the thermally driven inkjet print head in FIG. 1, which is used to manufacture a microarray according to the present invention.

Referring to FIGS. 2 and 3, the thermally driven inkjet print head includes a substrate 110, a nozzle plate 112 disposed on the substrate 110, and a heater 114 and an electrode 116, which are formed on the nozzle plate 121.

The substrate 110 may be formed of silicon, which is widely used to manufacture integrated circuits. A plurality of discharge chambers 106, which are semispherical and are filled of biomolecular solutions, are formed in a front surface of the substrate 110. A plurality of manifolds 102 in which the biomolecular solutions to be supplied to the discharge chambers 106 are stored are formed in a real surface of the substrate 110. The rear surface of the substrate 110 is covered with a glass plate (not shown) that has inlets through which the biomolecular solutions are injected into the respective manifolds 102.

A plurality of channels 104 connecting the discharge chambers 106 and the manifolds 102 are formed. The biomolecular solutions are supplied from the manifolds 102 to the discharge chambers 106 through the channels 104. The channels 104 are formed in a bottom center of the respective discharge chambers 106. The channels 104 may have a circular cross-section. The channels 104 may have a cross-section in any shape, for example, elliptical or polygonal shape.

The nozzle plate 112, which forms an upper wall portion of the discharge chambers 106, is stacked on the substrate 110. A plurality of nozzles 120 are formed in the nozzle plate 120 such as to correspond to the discharge chambers 106, particularly, center portions of the discharge chambers 106.

When the substrate 110 is made of silicon, the nozzle plate 112 may be formed of a silicon oxide layer obtained as a result of oxidizing the substrate 110. Alternatively, the nozzle plate 112 may be formed of a silicon nitride layer deposited on the substrate 110.

A plurality of heaters 114 are formed on the nozzle plate 112. The heaters 114 generate ink bubbles 130 by heating the biomolecular solutions contained in the discharge chambers 106. The heaters 114 have annular shapes and respectively surround the nozzles 120. The heater 144 is formed of a heating resistor such as doped polysilicon or a tantalum-aluminium alloy. In particular, the heaters 114 may be formed by depositing doped polysilicon or a tantalum-aluminium alloy over the nozzle plate 112 and patterning the deposited layer into an annular shape. The heaters 114 may be formed in any shape other than the annular shape.

A plurality of electrodes 166 that are electrically connected to the heaters 114 are formed on the nozzle plate 112. The electrodes 116 for supplying current as pulses to the heaters 114 are made of a metal such as aluminium or an aluminium alloy. The electrodes 116 are electrically connected to the pads 170 in FIG. 1 as described above.

A nozzle guide 122 extending toward the discharge chamber 106 is formed along a sidewall of each of the nozzles 120. The nozzle guide 122 formed along the sidewall of the nozzle 120 facilitates an ink droplet to be discharged straight.

Hereinafter, a process of spotting the biomolecular solutions stored in the discharge chambers 106 of the inkjet print head having the above structure onto a surface of the solid support 200 when manufacturing a microarray is described.

Initially, when current is supplied as pulses to the annular heaters 114 while the semispherical discharge chambers 106 are filled with the biomolecular solution, heat is generated by the heaters 114. The heat is transferred to the biomolecular solution in the discharge chambers 106 through the nozzle plate 112 underlying the heaters 114. As a result, the biomolecular solution under the heaters 114 boils. The bubbles resulting from the boiling have a doughnut shape depending on the shape of the annular heaters 114.

As the biomolecular solution is continuously heated, the bubbles 130 expand and combine to form larger bubbles having depressed inner surface and push the biomolecular solution out of the discharge chambers 106 through the nozzles 120.

When the bubbles 130 expand to a maximum level and the supply of current is interrupted, the bubbles 130 shrink and disappear. As a result, the discharge chambers 160 are under a negative pressure, and the bubbles 130 discharged through the nozzles 120 are spotted as droplets 150 onto the surface of the solid support 200.

The spots of the biomolecular solution are immobilized onto the surface of the solid support 200, thereby resulting in a complete microarray. The volume of a single droplet 150 spotted onto the surface of the solid support 200 is 25 pL. However, the volume of a single droplet can be varied according to the size of the nozzles 120 and the energy supplied to discharge the droplet.

When the bubbles 130 shrink and disappear, the discharge chambers 106 are refilled with the biomolecular solutions supplied through the channels 104 from the manifolds 102.

In the above-described process of spotting the biomolecular solution onto the solid support 200, the droplet 150 discharged through the nozzle 120 is sharply tailed as the doughnut-shaped bubbles 130 combine at the center of the discharge chamber 106 so that no satellite droplet is generated.

In addition, since the discharge chamber 106 is semispherical, the bubbles 130 are allowed to expand within the discharge chamber 106, thereby suppressing the biomolecular solution from flowing backward.

Since the heater 114 is annular, cooling and heating can be rapidly performed, and the time duration between the generation and disappearance of the bubbles 130 becomes short. Therefore, the thermally driven inkjet print head can be operated with high operation frequency. Furthermore, since the discharge chamber 106 is semispherical, the bubbles 103 can stably expand. Since the bubbles 130 can be rapidly generated and expand, the biomolecular solution can be discharged in a short time.

Due to the nozzle guide 122 formed along the sidewall of each of the nozzles 120, the droplet 150 of the biomolecular solution can be discharged exactly perpendicular to the surface of the solid support 200.

Hereinafter, whether or not biomolecules in the solution are influenced by the inkjet print head used to manufacture a microarray according to an embodiment of the present invention is described.

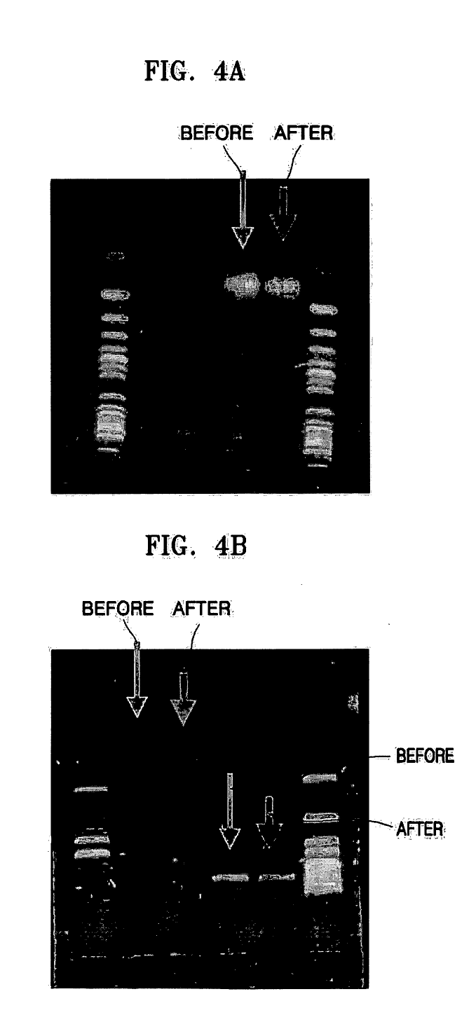

FIGS. 4A and 4B illustrate the results of gel electrophoresis on DNA before and after being discharged from the thermally driven inkjet print head used to manufacture a microarray according to an embodiment of the present invention. The results in FIG. 4A are obtained using 60 mer DNA as biomolecules, and the results in FIG. 4B are obtained using 300 mer DNA as biomolecules.

Referring to FIGS. 4A and 4B, no change occurs in the DNA after being discharged from the thermally driven inkjet print head used to manufacture a microarray according to the embodiment of the present invention.

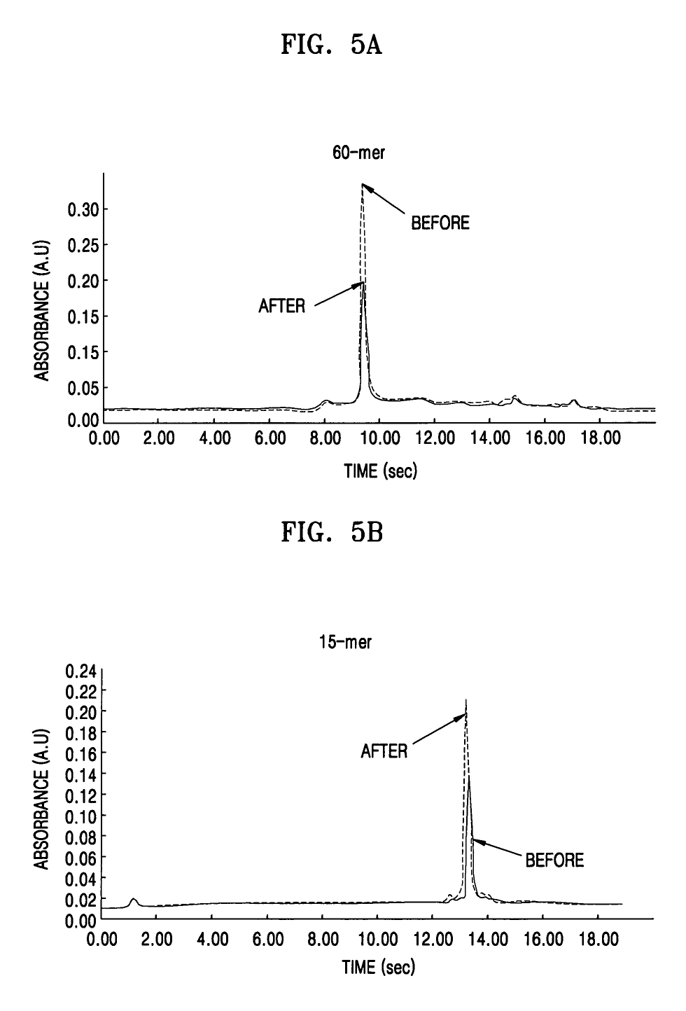

FIGS. 5A and 5B illustrate the results of analysing DNA using high performance liquid chromatography (HPLC) before and after being discharged from the thermally driven inkjet print head used to manufacture a microarray according to the present invention. The results in FIG. 5A are obtained using 60 mer DNA as biomolecules, and the results in FIG. 5B are obtained using 15 mer DNA as biomolecules.

Referring to FIGS. 5A and 5B, as when analysed using gel electrophoresis, no change occurs in the DNA after being discharged from the thermally driven inkjet print head used to manufacture the microarray according to the embodiment of the present invention.

Based on the above-described experimental results, it is apparent that biomolecules are not affected when discharged through the thermally driven inkjet print head used to manufacture a microarray according to the present invention.

As described above, a method and apparatus for manufacturing a microarray according to the present invention have the following effects.

First, a uniformly spotted microarray can be manufactured using a thermally driven inkjet print head according to the present invention because the thermally driven inkjet print head does not contact the microarray.

Second, the volume of biomolecular solution that is spotted at a time is less than in conventional methods.

Third, since the discharge chamber of the thermally driven inkjet print head that is filled with a biomolecular solution is semispherical, flowing backward of the biomolecular solution can be suppressed.

Fourth, microarrays can be rapidly manufactured on a large scale using the thermally driven inkjet print head.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of manufacturing a microarray by non-contact spotting a biomolecular solution onto a surface of a solid support using a thermally driven inkjet print head that comprises a discharge chamber filled with the biomolecular solution to be spotted and being substantially semispherical.

2. The method of claim 1, wherein the thermally driven inkjet print head comprises:
a substrate in which the discharge chamber, a manifold storing the biomolecular solution to be supplied to the discharge chamber, and a channel connecting the discharge chamber and the manifold are formed;
a nozzle plate disposed on the substrate and having nozzles through which the biomolecular solution is discharged from the discharge chamber;
a heater formed around each of the nozzles; and
an electrode electrically connected to the heater to supply current to the heater.

3. The method of claim 2, comprising:
supplying current to the heater through the electrode;
heating the biomolecular solution in the discharge chamber using heat generated by the heater to generate and expand bubbles; and
discharging the biomolecular solution from the discharge chamber onto the surface of the solid support through the nozzles by the expansive force of the bubbles.

4. The method of claim 2, wherein a plurality of discharge chambers, a plurality of manifolds, and a plurality of channels are formed in the substrate.

5. The method of claim 2, wherein the substrate is formed of silicon.

6. The method of claim 2, wherein the nozzle plate is formed of a silicon oxide layer or a silicon nitride layer.

7. The method of claim 2, wherein the heater has an annular shape and surround a corresponding nozzle in the nozzle plate.

8. The method of claim 2, wherein the heater is formed of polycrystalline silicon or a tantalum-aluminum alloy.

9. The method of claim 2, wherein the electrode is formed of aluminum or an aluminum alloy.

10. The method of claim 2, wherein a nozzle guide extending toward the discharging chamber is formed along a sidewall of each of the nozzles.

11. The method of claim 1, wherein the biomolecular solution contains an oligonucleotide as biomolecules.

12. The method of claim 1, wherein the biomolecular solution contains a protein as biomolecules.

13. The method of claim 1, wherein the biomolecular solution contains cDNA as biomolecules.

14. The method of claim 1, wherein the solid support is formed of glass.

15. The method of claim 1, wherein the solid support is formed of a silicon wafer.

16. The method of claim 1, wherein the surface of the solid support is coated with at least one material selected from the group consisting of amine, aldehyde, and epoxy.

17. A thermally driven inkjet print head for manufacturing a microarray by non-contact spotting a biomolecular solution onto a surface of a solid support, the thermally driven inkjet print head comprising:
a substrate in which a plurality of discharge chambers filled with the biomolecular solution to be spotted, a plurality of manifolds storing the biomolecular solution to be supplied to the discharge chambers, and a plurality of channels respectively connecting the discharge chambers and the manifolds are formed;
a nozzle plate disposed on the substrate and having a plurality of nozzles that respectively correspond to the discharge chambers;
a plurality of heaters respectively formed around the nozzles; and
a plurality of electrodes respectively electrically connected to the heaters to supply current to the heaters.

18. The thermally driven inkjet print head of claim 17, wherein the discharge chambers are substantially semispherical.

19. The thermally driven inkjet print head of claim 17, wherein the substrate is formed of silicon.

20. The thermally driven inkjet print head of claim 17, wherein the nozzle plate is formed of a silicon oxide layer or a silicon nitride layer.

21. The thermally driven inkjet print head of claim 17, wherein the heaters have annular shapes and respectively surround the nozzles in the nozzle plate.

22. The thermally driven inkjet print head of claim 17, wherein the heaters are formed of polycrystalline silicon or a tantalum-aluminum alloy.

23. The thermally driven inkjet print head of claim 17, wherein the electrodes are formed of aluminum or an aluminum alloy.

24. The thermally driven inkjet print head of claim 17, wherein a nozzle guide extending toward a corresponding discharging chamber is formed along a sidewall of each of the nozzles.

25. The thermally driven inkjet print head of claim 17, wherein the biomolecular solution contains an oligonucleotide as biomolecules.

26. The thermally driven inkjet print head of claim 17, wherein the biomolecular solution contains a protein as biomolecules.

27. The thermally driven inkjet print head of claim 17, wherein the biomolecular solution contains cDNA as biomolecules.

28. The thermally driven inkjet print head of claim 17, wherein the solid support is formed of glass.

29. The thermally driven inkjet print head of claim 17, wherein the solid support is formed of a silicon wafer.

30. The thermally driven inkjet print head of claim 17, wherein the surface of the solid support is coated with at least one material selected from the group consisting of amine, aldehyde, and epoxy.
